(19) United States
(12) Patent Application Publication
(10) Pub. No.: US 2015/0305645 A1
(43) Pub. Date: Oct. 29, 2015

(54) Off-Resonance Correction for Vessel-Selective Pseudo-Continuous Arterial Spin Labeling Imaging

(71) Applicants: KABUSHIKI KAISHA TOSHIBA, Tokyo (JP); Toshiba Medical Systems Corporation, Tochigi (JP)

(72) Inventors: Cheng OUYANG, Buffalo Grove, IL (US); Aiming LU, Chicago, IL (US); Mitsue MIYAZAKI, Des Plaines, IL (US)

(73) Assignees: KABUSHIKI KAISHA TOSHIBA, Tokyo (JP); Toshiba Medical Systems Corporation, Tochigi (JP)

(21) Appl. No.: 14/260,058

(22) Filed: Apr. 23, 2014

(51) Int. Cl. A61B 5/055; G01R 33/56; G01R 33/54; G01R 33/34; G01R 33/385

(52) U.S. Cl. CPC A61B 5/055; G01R 33/34084; G01R 33/385; G01R 33/543; G01R 33/56

(57) ABSTRACT

A magnetic resonance imaging (MRI) system, method and/or computer readable medium is configured to effect MR imaging based upon arterial spin labeling (ASL) by forming a plurality of ASL perfusion images of an object where each perfusion image corresponds to a respective phase offset, and by generating a corrected perfusion image by fitting corresponding points from each of the plurality of perfusion images to a polynomial function for respective points of the corrected perfusion image.

### FIELD

The subject matter below relates generally to magnetic resonance imaging (MRI). In particular, the subject matter relates to arterial spin labeling (ASL) and perfusion MRI.

### BRIEF DESCRIPTION OF THE DRAWINGS

FIG. 1 is a high-level schematic block diagram of an MRI system adapted for improved perfusion MRI, in accordance with one or more embodiments.

FIG. 2 illustrates a conventional pulse sequence used for vessel-selective pseudo-continuous ASL imaging.

FIG. 3 illustrates a flowchart for off-resonance correction of perfusion territory images, in accordance with one or more embodiments.

FIG. 4 illustrates a tagging pulse sequence and in-plane gradients in accordance with one or more embodiments to illustrate a sequence scheme of vessel-selective pCASL with off-resonance correction where Θ is the phase related to the original vessel-selective pCASL sequence and Δψ is the extra phase offset added to the sequence.

FIG. 5 illustrates a curve fitted to simulated inversion efficiencies acquired for respective phase offsets for a particular voxel, in accordance with one or more embodiments to illustrate a simulated inversion efficiency (velocity 30 cm/s) of vessel-selective pCASL at different phase offsets (small circles) and the solid line is the 12^{th} order polynomial fitted to the simulations.

FIG. 6 illustrates a set of images including uncorrected and corrected perfusion-weighted images, according to one or more embodiments by illustrating a) the overlay magnitude image of the labeling slice where the dotted circle stands for the target artery of the right ICA; b) the labeling pattern obtained in vivo at the labeling slice shown in a) where the dotted circle delineates the size and position of the single-artery labeling disk; c) the measured regional control-tag data with different phase offsets; d) the estimated regional CBF-weighted map; and e) the estimated phase error (in degree units) map.

### DETAILED DESCRIPTION

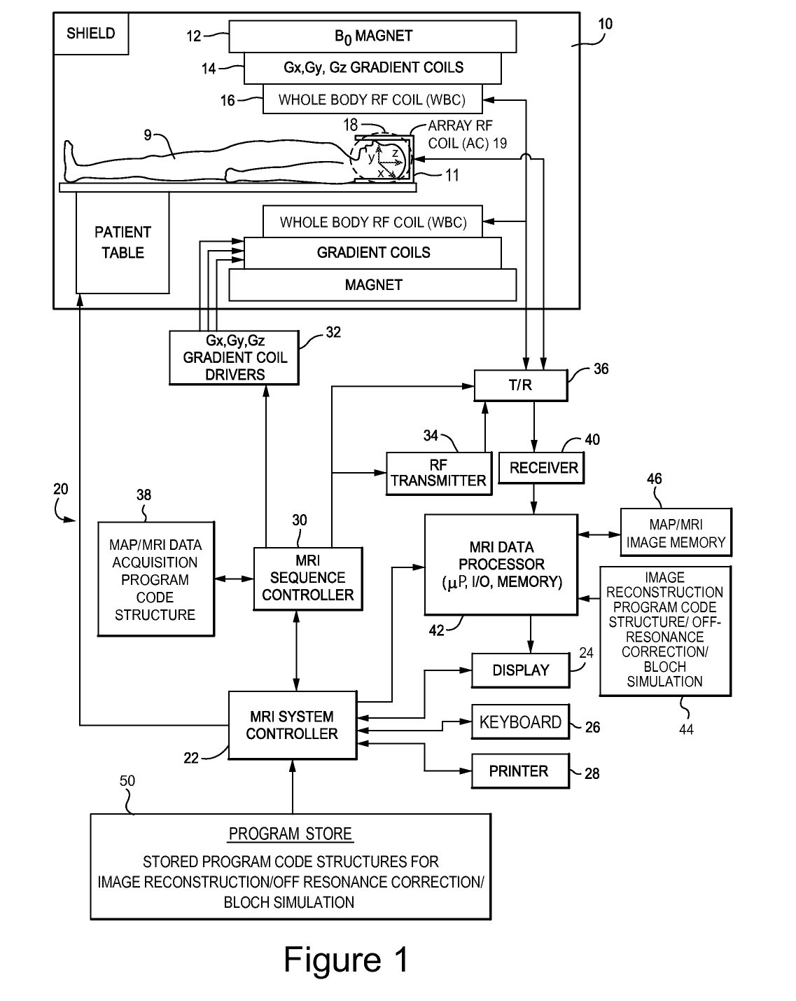

The MRI system shown in FIG. 1 includes a gantry 10 (shown in schematic cross-section) and various related system components 20 interfaced therewith. At least the gantry 10 is typically located in a shielded room. The MRI system geometry depicted in FIG. 1 includes a substantially coaxial cylindrical arrangement of the static field B₀ magnet 12, a Gx, Gy and Gz gradient coil set 14 and a large whole body RF coil (WBC) assembly 16. Along the horizontal axis of this cylindrical array of elements is an imaging volume 18 shown as substantially encompassing the head of a patient 9 supported by a patient table 11. Smaller array RF coils 19 might be more closely coupled to the patient head in imaging volume 18. As those in the art will appreciate, compared to the WBC (whole body coil), relatively small coils and/or arrays such as surface coils or the like are often customized for particular body parts (e.g., arms, shoulders, elbows, wrists, knees, legs, chest, spine, etc.). Such smaller RF coils are herein referred to as array coils (AC) or phased array coils (PAC). These may include at least one coil configured to transmit RF signals into the imaging volume and a plurality of receiver coils configured to receive RF signals from an object, such as the patient head in the example above, in the imaging volume.

An MRI system controller 22 has input/output ports connected to a display 24, keyboard 26 and printer 28. As will be appreciated, the display 24 may be of the touch-screen variety so that it provides control inputs as well and a mouse or other I/O device(s) may be provided.

The MRI system controller 22 interfaces with MRI sequence controller 30 which, in turn, controls the Gx, Gy and Gz gradient coil drivers 32, as well as the RF transmitter 34 and the transmit/receive switch 36 (if the same RF coil is used for both transmission and reception). The MRI sequence controller 30 includes suitable program code structure 38 for implementing MRI imaging (also known as nuclear magnetic resonance, or NMR, imaging) techniques, which may also include parallel imaging. As described below, sequence controller 30 may be configured to apply a predetermined tagging pulse sequence and a predetermined control pulse sequence, in order to obtain corresponding tagging and control images from which a diagnostic MRI image is obtained. MRI sequence controller 30 may also be configured for EPI imaging and/or parallel imaging. Moreover, MRI sequence controller 30 may facilitate one or more preparation scan (prescan) sequences, and a scan sequence to obtain a main scan MR image (sometimes referred to as a diagnostic image).

The MRI system 20 includes an RF receiver 40 providing input to data processor 42 so as to create processed image data, which is sent to display 24. The MRI data processor 42 is also configured for access to previously generated MR data, images, and/or maps, and/or system configuration parameters 46 and MRI image reconstruction program code structures 44 and 50.

Also illustrated in FIG. 1 is a generalized depiction of an MRI system program store 50 where stored program code structures (e.g., for image reconstruction of control and tagging images, for generation of subtracted image, etc. as described below, for simulation of selected MRI image characteristics, for post-processing MRI etc.) are stored in non-transitory computer-readable storage media accessible to the various data processing components of the MRI system. As those in the art will appreciate, the program store 50 may be segmented and directly connected, at least in part, to different ones of the system 20 processing computers having most immediate need for such stored program code structures in their normal operation (i.e., rather than being commonly stored and connected directly to the MRI system controller 22).

Indeed, as those in the art will appreciate, the FIG. 1 depiction is a very high-level simplified diagram of a typical MRI system with some modifications so as to practice exemplary embodiments described hereinbelow. The system components can be divided into different logical collections of “boxes” and typically comprise numerous digital signal processors (DSP), microprocessors and special purpose processing circuits (e.g., for fast A/D conversions, fast Fourier transforming, array processing, etc.). Each of those processors is typically a clocked “state machine” wherein the physical data processing circuits progress from one physical state to another upon the occurrence of each clock cycle (or predetermined number of clock cycles).

Not only does the physical state of processing circuits (e.g., CPUs, registers, buffers, arithmetic units, etc.) progressively change from one clock cycle to another during the course of operation, the physical state of associated data storage media (e.g., bit storage sites in magnetic storage media) is transformed from one state to another during operation of such a system. For example, at the conclusion of an image reconstruction process and/or sometimes the generation of a subtracted image from control and tagging images, as described below, an array of computer-readable accessible data value storage sites in physical storage media will be transformed from some prior state (e.g., all uniform “zero” values or all “one” values) to a new state wherein the physical states at the physical sites of such an array vary between minimum and maximum values to represent real world physical events and conditions (e.g., the internal physical structures of a patient over an imaging volume space). As those in the art will appreciate, such arrays of stored data values represent and also constitute a physical structure—as does a particular structure of computer control program codes that, when sequentially loaded into instruction registers and executed by one or more CPUs of the MRI system 20, causes a particular sequence of operational states to occur and be transitioned through within the MRI system.

Arterial spin labeling (ASL) is an MRI technique that is of particular interest for perfusion and non-contrast enhanced MRA applications. ASL relies upon the inflow of blood into the volume being imaged, and uses separate control and tag pulse sequences to label (i.e., tag) spins of inflowing blood differently. Separate images are generated based upon the control pulse sequence and the tag pulse sequence. An image generated based upon a control pulse sequence is referred to as a “control image,” and an image generated based upon a tag pulse sequence is referred to as a “tag image.” A perfusion MRA image can be obtained by subtracting the tag image from the control image.

Dai et al., “Continuous Flow-Driven Inversion for Arterial Spin Labeling Using Pulsed Radio Frequency and Gradient Fields,” Magnetic Resonance in Medicine 60:1488-1497 (2008), describes pseudo-continuous arterial spin labeling (pCASL) which is used frequently for many applications including intracranial applications. However, its tagging efficiency is highly sensitive to off-resonance effects and gradient imperfections, which induce phase mismatches or phase errors between the radiofrequency pulses (Wu et al., Magnetic Resonance in Medicine 58:1020-27 (2007)). This sensitivity can lead to tagging efficiency loss, signal to noise ratio (SNR) loss, and unpredictable variations in acquired perfusion images. The high sensitivity may be due, at least in part, to the tag and control conditions of flowing arterial blood being, to a significant extent, defined by the specification of the phases in the RF pulse train. Jung et al., “Multiphase Pseudocontinuous Arterial Spin Labeling (MP-PCASL) for Robust Quantification of Cerebral Blood Flow,” Magnetic Resonance in Medicine 64:799-810 (2010), described a variation of pCASL that may have reduced the sensitivity to off-resonance artifact.

Regional Perfusion Imaging (RPI) based on ASL provides the ability to noninvasively delineate the perfusion territories of major cerebral arteries. Rather than injecting a flow tracer, ASL employs RF and magnetic field gradient pulses to invert naturally existing water spins in the feeding arteries. Many ASL techniques including pCASL and MP-PCASL noted above, however, label all the arteries feeding the perfusion region.

Several ASL techniques have been proposed for observing individual perfusion territories. The general principle of these RPI techniques is to tag only arterial spins flowing through the artery or arteries of interest, while avoiding the tagging of spins in other arteries. Control over which arteries are labeled can be used to measure the tissue regions that are perfused by particular vessels (e.g., arteries) and to characterize the dynamics of flow through vessels, occlusions, arteriovenous malformations, aneurysms, and the like.

In some applications, the delineation of perfusion territories by RPI provides complementary information to angiography, such as, for example, information regarding the status of blood flow in different regions of the arterial tree.

Dai et al., “Modified Pulsed Continuous Arterial Spin Labeling for Labeling a Single Artery,” Magnetic Resonance in Medicine 64:975-982, 2010 (hereafter “Dai VS-pCASL”) which is herein incorporated by reference in its entirety, describes one or more techniques for modifying pCASL RF pulse sequences to selectively map vascular territories of major cerebral feeding arteries. In the vessel-selective, or single-artery, pCASL approach (VS-pCASL), RPI is accomplished by inserting additional in-plane gradients in the gaps between discrete RF pulses to modulate the phases of flowing spins in different vessels in the labeling plane.

RPI can be a very useful clinical tool to investigate several cerebrovascular disorders or diseases, such as, for example, occlusion in internal carotid arteries (ICAs) (Hendrikse et al., Neurosurgery 57:486-96 (2005); van Laar et al., Radiology 242:526-34 (2007)), arteriovenous malformations (Fiehler et al., AJNR Am J Neuroradiol 30:356-61 (2009)), and collateral flow between major arteries (Hendrikse et al., Stroke 35:882-7 (2004)).

However, similar to pCASL, VS-pCASL too is vulnerable to off-resonance effects, which can cause degradation in vessel-selective tagging efficiency and failure in vessel-selective perfusion imaging. Consequently, this sensitivity may compromise the application of VS-pCASL in clinical settings.

Loss in vessel-selective tagging efficiency can be especially true in experiments to separate perfusion regions of left, right ICAs and vertebral artery, where off-resonance effects (e.g., strong field inhomogeneities) are usually observed at the labeling plane around the neck. Such inhomogeneities can also be a concern when imaging perfusion territories of smaller arterial branches, such as Circle-of-Willis (COW) branches. For example, in some brain regions, such as the orbital frontal cortex, significant magnetic field inhomogeneity artifacts exist due to their close proximity of tissue/air boundaries (Truong et al., “Three-dimensional numerical simulations of susceptibility-induced magnetic field inhomogeneities in the human head,” Magnetic Resonance in Medicine 20:759-70 (2002)). The vessel-selective labeling can be seriously contaminated if the labeling plane of the target artery (for example, arterial cerebral arteries) passes through these regions.

A careful manual shimming before VS-pCASL tagging may improve the main field homogeneity and lessen the influence of off-resonance effects; however, in practice, sufficient field homogeneity cannot be achieved by shimming alone.

In short, the single-artery, or vessel-selective, pCASL sequence has been demonstrated to provide regional perfusion maps non-invasively. However, similar to the original pCASL labeling, vessel-selective pCASL is also observed to be vulnerable to off-resonance effects, which introduce phase errors in the labeling RF train and thus cause degradation in tagging efficiency. Below, we propose to restore the signal loss due to off-resonance artifacts by applying a modified multiple phase correction method in the vessel-selective labeling sequence.

Embodiments described in the present application include novel schemes to restore the signal loss due to off-resonance artifacts by applying a phase correction technique in the VS-pCASL labeling sequence or other territory-selective ASL-based sequences. Embodiments provide for estimating the phase offsets or phase errors at the target feeding artery, and effectively restoring the corresponding signal loss due to off-resonance artifact. In this manner, some embodiments provide higher SNR and more robust measurements in VS-pCASL or other territory-selective ASL-based sequences.

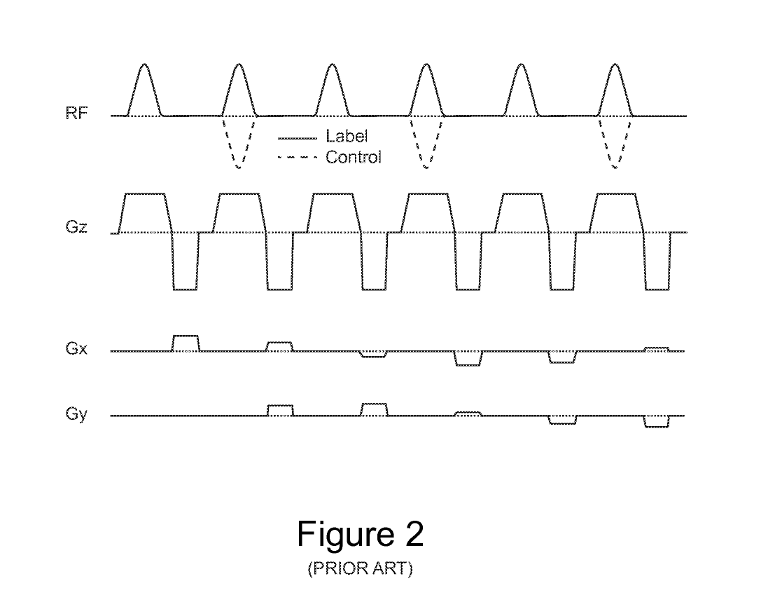

FIG. 2 illustrates a single artery selective pseudocontinuous sequence 200 described in Dai VS-pCASL. The labeling technique described Dai VS-pCASL takes as input a specification of a target vessel position and adds rotating in-plane gradients to the pCASL pulse sequence to achieve localized labeling while spoiling undesired labeling of other vessels.

As illustrated, the tagging pulse train comprises equally-spaced RF pulses. A small imbalance in the gradients along the flow direction is added for tagging. In the control pulse train, the RF pulses are equally-spaced but maintain a 180-degree phase shift between consecutive pulses.

Specifically, in order to achieve vessel-selectivity, in addition to the labeling gradient along the flow direction as used in pCASL, VS-pCASL introduces in-plane gradients between the RF pulses which produce a phase shift between vessels. The direction of the in-plane gradients is then rotated as illustrated in FIG. 2, in order to achieve the single vessel selectivity.

VS-pCASL results in the selective labeling of a disk, the center of which is on a target vessel. The center of the disk is controlled by the phases of the RF pulses. The phase of each RF pulse is incremented in phase relative to the pulse immediately before it by an angle determined based upon the applied gradients and the desired disk center. Dai VS-pCASL provides techniques for calculating the phases for VS-pCASL pulse sequence.

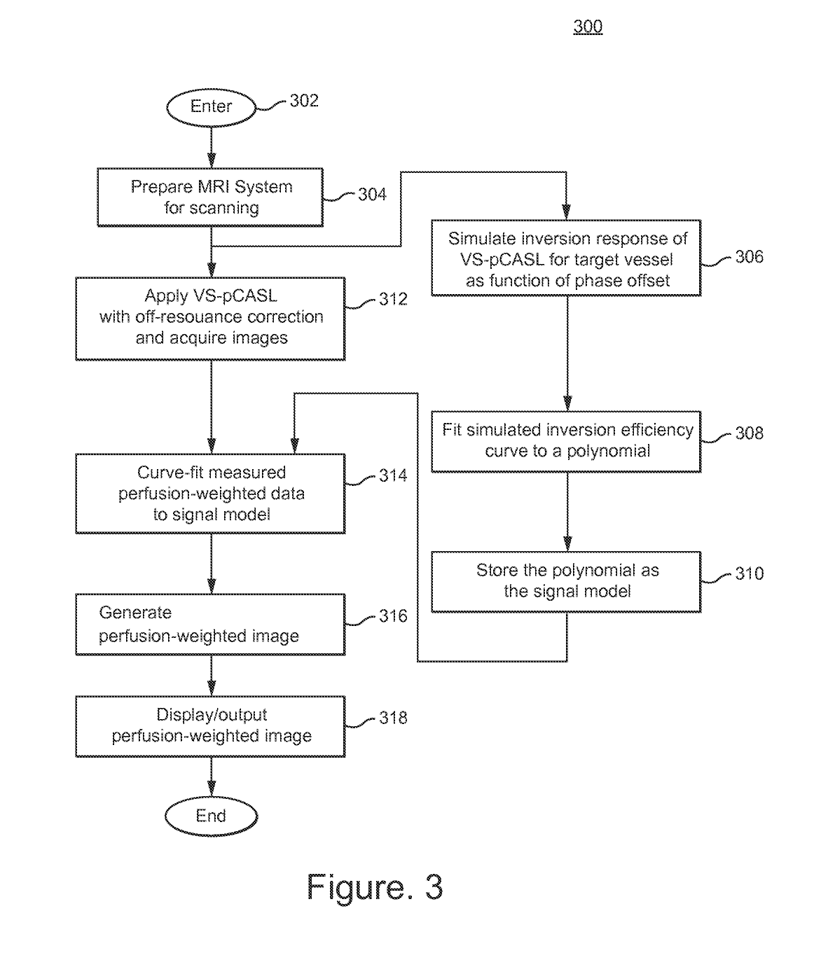

FIG. 3 illustrates a flowchart for a process 300 for off-resonance correction of ASL-based perfusion images, in accordance with one or more embodiments. The process 300 may be performed by a MRI system, such as, for example, the MRI system shown in FIG. 1. It will be appreciated that one or more of the operations 304-318 may be performed in an order other than that shown, may not be performed or may be combined with one or more other operations when performing process 300.

At operation 302, process 300 for off-resonance correction of ASL-based perfusion images is entered. The MRI system and the patient are then, at operation 304, prepared for scanning. Operation 304 may include positioning the patient and/or the part of the patient to be imaged in relation to transmit and/or receive coils of the MRI system, and setting of general parameters and/or configuration options for performing imaging.

The techniques described herein can be applied to image many parts of the patient, such as, but not limited to, head, neck, knee, or other area, with appropriate configurations of the system and positioning of the patient. As described below, certain configurations, such as, for example, tagging and/or control slab locations, tagging slice thickness, the number of tagging pulses, a total duration of tagging, and time delay between tagging pulses can be adjusted in a respective manner based upon selected characteristics of the object image. For example, configurations may be set and/or adjusted in accordance with the flow speed of the vessel or specific part of the body or organ being imaged. Other configurations may include specifying a vessel or vessels (e.g., in a head or neck scanning application, the left or right ICA) in which the blood is to be tagged.

The preparation stage may, in some embodiments, also include acquiring one or more prescans, for example, to obtain one or more low resolution MRI images for positioning the patient, coil calibration, locating tagging and/or control slabs/planes, and/or to determine the position of the vessel(s) identified for tagging.

At operation 306, the inversion response of the ASL technique for the target vessel as a function of phase offset is simulated. The “inversion response” represents the ratio of the net magnetization along the z-axis (e.g., obtained by subtracting control image—tag image) to the magnetization of relaxed blood. Simulations may be performed to obtain values for the inversion responses of VS-pCASL labeling at the target vessel as a function of phase offset (i.e., ALP discussed below). Simulated inversion responses from an example simulation are shown as small circles in FIG. 5.

The simulation may be provided with initial parameters for properties (e.g., shape, width, spacing between pulses, amplitude of pulses, number of pulses, flip angle, phase, etc.) for tagging and control RF pulses, gradient parameters (e.g., Gₓ, G_{y}, G_{z}, average gradient strength for each gradient, amplitude, etc.). Other parameters may also include tagging plane, control plane, vessel(s) to be tagged, and imaging plane configurations. Yet other parameters provided may include in-plane gradient rotation rates, and in-plane gradient rotation pattern (e.g., Gₓ as a particular sine curve and G_{y} as a particular cosine curve) which may be used for vessel-selective tagging.

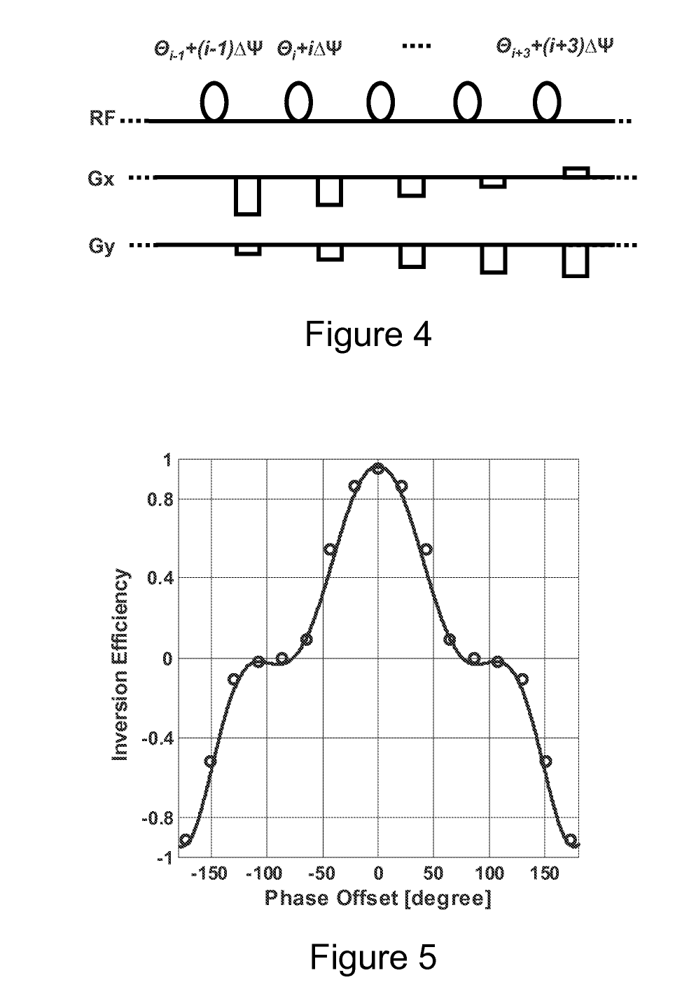

According to an embodiment, numerical Bloch simulations are performed to determine the simulated inversion responses (control-tag) of the vessel-selective pCASL labeling at the target vessel as a function of phase offset (Δψ as shown in FIG. 4). The simulation may be implemented in MATLAB (MathWorks Inc., Natick, Mass.) or similar tool.

At operation 308, the simulated inversion responses are fitted to a polynomial. According to an embodiment, the simulated inversion response curve was fitted to a 12^{th} order polynomial P(Δψ), shown in FIG. 5 (e.g., where y=ax¹²+bx¹¹+cx¹⁰+dx⁹+ . . . +mx+1). The curve fitting may be based upon any known technique, such as, but not limited to, the least squares method, or minimum root-mean-square error.

At operation 310, the polynomial is stored to be subsequently used as the signal model for the correction process where the perfusion signal can be estimated by fitting the measured perfusion-weighted data (m_{i,n}) at multiple phase offsets to the expected inversion efficiency function in a voxel-by-voxel manner as shown in equation (1) where CBF is the perfusion-weighted map, and ε is the phase error map. In embodiments, the availability of a polynomial, or more specifically a high order polynomial such as, but not limited to, a 12^{th} order polynomial, as a signal model may provide a better fit than other types of functions that may be fitted to the simulated data points, as more free variables are available in the polynomial fit.

Operation 306 may or may not be performed during the scanning process. In some embodiments, the simulation may be performed entirely, or in part, offline from the scanning, and the results uploaded to the MRI system. In other embodiments, the simulation may be performed on-line, for example, by accessing configuration parameters (e.g., pulse configurations, vessel selection etc.) automatically, either during or after the preparation processing of operation 304.

At operation 312, the RF pulse sequence with off-resonance correction is applied. According to an embodiment, the applied RF pulse sequence is the VS-pCASL pulse sequence modified to include off-resonance correction. The original VS-pCASL is illustrated in FIG. 2. Amplitude-varying in-plane gradients are included between any two RF pulses in the pCASL RF pulse train to create a rotating phase distribution across the labeling plane (e.g., around the vessel selected to be tagged) and to spoil the labeling of unselected feeding arteries. Consequently, a selected vessel may be tagged without tagging other vessels in the same labeling plane.

FIG. 4 is an illustration of the tagging pulse sequence modified to include off-resonance correction according to one or more embodiments. FIG. 4 illustrates only the tagging pulse train and the in-plane gradients. As illustrated, the tagging sequence includes a train of equally-spaced RF selective pulses similar to the VS-pCASL pulse sequence shown in FIG. 2. Also, as in FIG. 2, in-plane rotating gradients are provided in between every pair of RF pulses. The gradient in the flow direction and the control pulse sequence is not shown in FIG. 4. Moreover, as shown in FIG. 4, in some embodiments, one or more additional phase offsets Δψ is added to each pulse in the sequence for off-resonance considerations. Θ represents the phase related to the basic VS-pCASL sequence (i.e., VS-pCASL without the off-resonance correction).

Returning to FIG. 3, the configured tagging pulse sequence and control pulse sequence are applied at 312. The train of tagging pulses of the tagging pulse sequence is applied to the tagging area (usually upstream from the imaging area so that the tagged blood will flow into the imaging area after a delay), and the train of control pulses in the control pulse sequence is applied to the control area respectively. The control pulse works as a pair with the tagging pulse, and is applied for each tagging pulse in order to cancel the inhomogeneous MT effect within the imaging slab.

Operation 312 may also include acquisition of images. In some embodiments, the tagging pulse sequence and the control pulse sequence are each configured with an imaging pulse train. Thus, in some embodiments, the tagging pulse sequence includes a train of tagging pulses and a train of imaging pulses; and the control pulse sequence includes a train of control pulses and a train of imaging pulses.

According to an embodiment, an imaging sequence follows each tagging pulse train and each control pulse train. The imaging may be performed according to a predetermined imaging pulse such as, but not limited to, 2D/3D Field Echo (FE), Fast Field Echo (FFE), Fast Spin Echo (FSE), Steady State FSE (SSFSE), Balanced Steady-State Free Precession (bSSFP), Ultrashort Echo Time (UTE), etc., imaging pulse sequences. In one or more embodiments, the imaging pulse trains in the tagging pulse sequence and the control pulse sequence may be identical.

Images may be acquired for a plurality of phase offsets. In an embodiment, separate images are acquired for phase offsets Δψ=−120°, −90°, −60°, −30°, 0°, 30°, 60°, 90°, and 120°. Acquiring an image at a particular phase offset may include acquisition of corresponding tag and control images, and the subtraction of the tag from the control image. In one or more embodiments, a predetermined number (e.g., the number of unique phase offsets configured for imaging) of separate images are acquired with each image corresponding to a unique phase offset. The acquired images represent uncorrected perfusion-weighted images.

At operation 314, the measured perfusion data is fitted to the signal model discussed in relation to operation 310. The measured perfusion data is obtained from the uncorrected perfusion-weighted images. The fitting of the measured perfusion data to the signal model may be performed on a pixel-by-pixel basis of a yet to be created corrected perfusion-weighted image. Operation 316 may be considered a post-processing activity to the extent that it is performed after the NMR data acquisition has been completed.

The curve fitting at operation 314 may include, for each voxel i in the yet to be formed corrected perfusion-weighted image, having a plurality of measured values m_{i,n} where n ranges from 1 to the number of images acquired. According to an embodiment, a separate image is acquired for each unique phase offset from a predetermined set of phase offsets. For example, separate images may be acquired for each of −120°, −90°, −60°, −30°, 0°, 30°, 60°, 90° and 120° phase offsets, yielding a total of nine images that can contribute to voxel i, which may be represented as m_{i, n} where n=1 . . . 9. In this example, the curve fitting includes fitting m_{i, n} to the signal model. The curve fitting may be performed by any appropriate curve fitting technique. According to an embodiment, the curve fitting may be performed according to a minimum root-mean-square error technique.

Due to off-resonance artifact in the labeling plane and/or arterial blood, measured values m_{i,n}, may be shifted in phase relative to the signal model. The amount of phase shift or phase offset of m_{i,n} from the signal model for i may be represented as εᵢ.

The measured perfusion-weighted data m_{i,n} may be represented as in the following equation (1): m_{i,n}=CBFᵢ×P(Δψₙ−εᵢ), where P is the signal model, and εᵢ is the phase error or phase offset determined by the curve fitting. CBFᵢ is corrected cerebral blood flow value for voxel i (also referred to as a corrected perfusion-weighted map value). Then, because m_{i,n} is known from the measurement and P(Δψₙ−εᵢ) is known from the fitted curve, CBFᵢ and εᵢ can be determined from equation (1). It should be noted that, after the curve fitting of m_{i,n} is performed, CBFᵢ is not dependent on either the number of phase offsets or the measured value at a particular phase offset.

At operation 316, the corrected perfusion-weighted image (CBFᵢ) is generated, and at operation 318, the obtained corrected perfusion-weighted image may be output to a display, to storage, directed to a printer, or communicated to another device for further processing. According to an example embodiment, the corrected perfusion-weighted image may be used to view a tissue region of interest in which the perfused blood delivered from a selected artery is clearly shown.

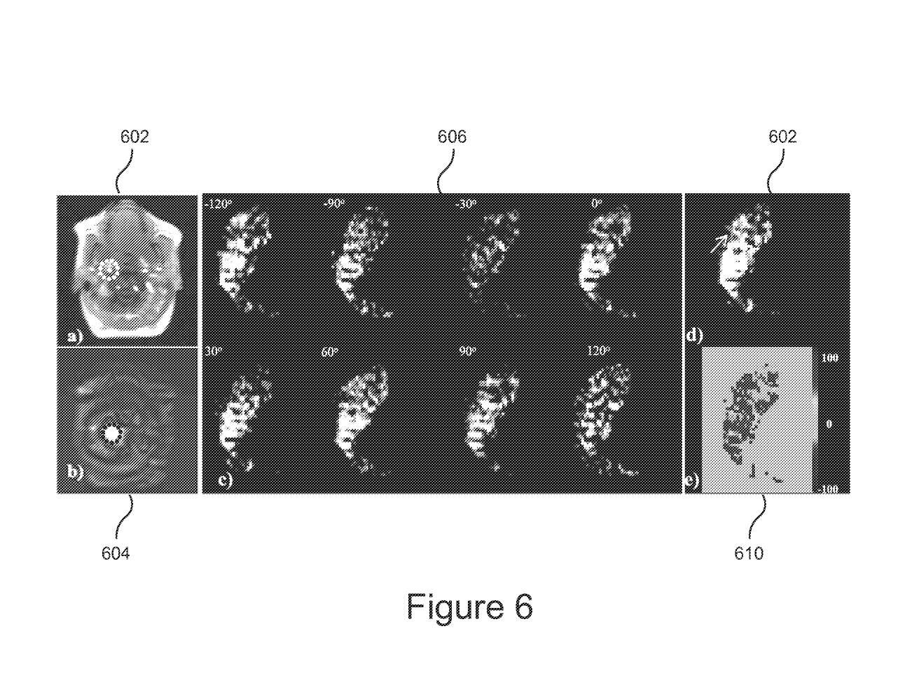

FIG. 6 illustrates some images representing an example embodiment. The measured data for the images shown in FIG. 6 were obtained by acquiring a respective image at each of a plurality of phase offsets n=1:8, Δψ=−120°, −90°, −30°, 0°, 30°, 60 °, 90°, 120°. The data acquired at the phase offset of −60° was discarded due to extensive motion artifact. One or more images may be output, for example, at operation 318.

In FIG. 6, (a), 602, represents an example overlay image acquired at the labeling slice depicting the feeding arteries (e.g., the dotted circle delineates the location of the right ICA), and (b), 604, illustrates the example vessel-selective tagging pattern obtained at the labeling location with phase offset of 0° (e.g., the dotted circle in (b) delineates the labeling disk around the right ICA). The labeling disk is expected to be a smooth circle under ideal conditions. The irregular edge of the labeling disk in FIG. 6b) can be caused by Bₒ inhomogeneities and other off-resonance effects.

The measured perfusion-weighted data at different phase offsets (e.g., m_{i,n}) is shown in (c), 606. Images (d), 608, and (e), 610, illustrate the estimated CBF-weighted and phase error maps, separately. By setting the estimated CBF signal level (e.g., image (d)) to 1.0, the mean absolute signal levels at each phase offset of the right ICA were observed to yield 0.79, 0.59, 0.28, 0.68, 0.65, 0.73, 0.61, and 0.41 in the order as shown in (c), thus illustrating the enhancement of the off-resonance corrected signal. As will be seen, the pattern of signal changes with different phase offsets is consistent with the simulation results. The SNR was improved by 47% by the proposed correction method compared to the signal obtained at 0° phase offset.

In the illustrated embodiments, the parameters used in simulation and in vivo experiments were: hamming-shaped RF pulses with 600 μs duration, 1.8 mm tagging slice thickness, gradient fraction 0.1, RF spacing 1500 μs, in-plane vessel-selective gradient amplitude 0.7 mT/m, gradient rotation rate of 11°, blood velocity 30 cm/s, tag duration 1.5 s, post-labeling delay 1 s with background suppression. A T₂ of 275 ms and a T₁ of 1680 ms was used in the simulation. One healthy subject was scanned in Toshiba® 3T Titan magnet, FFE2D readout (FA/TR/TE: 20 0/9/3.4 ms, matrix size 642, imaging slice thickness 10 mm, total TR 6 s, single slice). Three averages at each phase offset and eight offsets (n-1:8, −120 °, −90°, −30°, 0°, 30°, 60°, 90°,120°) were obtained, resulting in an acquisition time of around 4.5 minutes. The data acquired at the phase offset of −60° was discarded due to extensive motion artifact.

Thus, embodiments effectively restore the signal loss due to off-resonance artifact in vessel-selective pCASL and thus provide higher SNR compared to original VS-pCASL. Embodiments may also yield improved signal to noise ratio (SNR), for example, when compared to the signal without correction obtained, for example, at 0° phase offset.

Although the above embodiments were described primarily with respect to the VS-pCASL technique, the teachings herein are applicable for off-resonance correction of other territory-selective ASL techniques such as, for example, and without limitation, Ouyang et al., “Regional Perfusion Imaging Using pTILT,” Journal of Magnetic Resonance Imaging, doi: 10.1002/jmri.24346 (2013).

As demonstrated by both simulated and human results, the efficiency of vessel-selective pCASL labeling can be degraded in the presence of off-resonance effects. However, as demonstrated above, the proposed modified multiple-phase correction method can effectively restore signal loss due to off-resonance artifact and thus provide higher SNR in vessel-selective pCASL. Another benefit to application of the multiple phase correction method in single-artery pCASL is that, unlike the non-vessel-selective pCASL sequence, for the single-artery labeling, the signal model shown in FIG. 5 or in equation (1) is still correct under the scenario of blood mixing, and the off-resonance correction does not compromise accuracy. Future enhancements to incorporating off-resonance correction into vessel-selective pCASL may further improve temporal resolution and SNR efficiency.

While certain embodiments have been described, these embodiments have been presented by way of example only and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A magnetic resonance imaging (MRI) system for effecting MR imaging based upon arterial spin labeling (ASL), said MRI system comprising:
an MRI gantry including a static magnetic field coil, gradient magnetic field coils, at least one radio frequency (RF) coil configured to couple with an object located in an imaging volume;
an MRI sequence controller configured to perform an RF and gradient magnetic field pulse sequence comprising (1) applying a tagging pulse train to a tagging area located upstream from an imaging area, followed by applying a first imaging pulse train to the imaging area, and (2) applying a control pulse train to a control area followed by applying a second imaging pulse train to the imaging area; and
at least one digital data processor configured to:
receive a plurality of first digital data and a plurality of second digital data corresponding respectively to nuclear magnetic resonance (NMR) signals responsive to the first imaging pulse train and to NMR signals responsive to the second imaging pulse train;
form, from each of the plurality of first digital data, a respective tag image and, from each of the plurality of second digital data, a respective control image;
form a plurality of perfusion images of the object, each of the perfusion images formed by one of the tag images and a corresponding one of the control images, each perfusion image corresponding to a respective phase offset;
generate a corrected perfusion image by, for respective points of the corrected perfusion image, fitting corresponding points from each of the plurality of perfusion images to a polynomial function; and
output the corrected perfusion image to a display, or data storage in a non-transient digital data storage medium, or an outbound data transmission port.

2. The MRI system of claim 1, wherein the MRI sequence controller is further configured to include, in the tagging pulse train, RF pulses having different phase offsets.

3. The MRI system of claim 1, wherein the tagging pulse train is configured to selectively tag a part of a corresponding tagging plane.

4. The MRI system of claim 3, wherein the tagging pulse train is configured to selectively tag one of a plurality of blood carrying vessels in a labeling plane.

5. The MRI system of claim 1, further comprising:
determine, by simulation, inversion response values associated with the tagging pulse train at the object as a function of phase offsets; and
fit the inversion response values determined by simulation to the polynomial function.

6. The MRI system of claim 5, wherein the polynomial function is a twelfth-order polynomial.

7. The MRI system of claim 1, wherein the said correcting is performed for respective voxels in the corrected perfusion image.

8. The MRI system of claim 7, wherein a value at a particular voxel in the corrected perfusion image is determined based upon a value of the particular voxel in the perfusion image and the polynomial.

9. The MRI system of claim 8, wherein the value at the particular voxel in the corrected perfusion image is determined according to m_{i,n}=vᵢ×P(Δψₙ−εᵢ), wherein vᵢ is the value at the particular voxel in the corrected perfusion image, m_{i,n} is a value of the particular pixel in the perfusion image, and P(Δψₙ−εᵢ) is the fitted polynomial function.

10. The MRI system of claim 1, wherein the tagging pulse train comprises a set of evenly spaced RF pulses, respective ones of the RF pulses including phase corrections from the multiple phase offsets.

11. The MRI system of claim 10, wherein amplitude-varying in-plane gradients are added between consecutive RF pulses.

12. The MRI system of claim 11, wherein the tagging pulse train corresponds to a vessel-selective pseudo-continuous arterial spin labeling (VS-pCASL).

13. A magnetic resonance imaging (MRI) method for effecting MR imaging based upon arterial spin labeling (ASL), said MRI method comprising:
placing an object into an MRI gantry including a static magnetic field coil, gradient magnetic field coils, at least one radio frequency (RF) coil configured to couple with an object located in an imaging volume;
performing an RF and gradient magnetic field pulse sequence comprising (1) applying a tagging pulse train to a tagging area located upstream from an imaging area, followed by applying a first imaging pulse train to the imaging area, and (2) applying a control pulse train to a control area followed by applying a second imaging pulse train to the imaging area;
receiving a plurality of first digital data and a plurality of second digital data corresponding respectively to nuclear magnetic resonance (NMR) signals responsive to the first imaging pulse train and to NMR signals responsive to the second imaging pulse train;
forming, from each of the plurality of first digital data, a respective tag image and, from each of the plurality of second digital data, a respective control image;
forming a plurality of perfusion images of the object, each of the perfusion images formed by one of the tag images and a corresponding one of the control images, each perfusion image corresponding to a respective phase offset;
generating a corrected perfusion image by, for respective points of the corrected perfusion image, fitting corresponding points from each of the plurality of perfusion images to a polynomial function; and
outputting the corrected perfusion image to a display, or data storage in a non-transient digital data storage medium, or an outbound data transmission port.

14. A non-transitory computer readable storage medium, having executable computer program instructions recorded thereon, which when executed by at least one processor of a magnetic resonance imaging (MRI) system having an MRI gantry including a static magnetic field coil, gradient magnetic field coils, at least one radio frequency (RF) coil configured to couple with an object located in an imaging volume, causes the at least one processor to generate a final MRI image, by performing operations comprising:
configuring a sequence controller to perform an RF and gradient magnetic field pulse sequence comprising (1) applying a tagging pulse train to a tagging area located upstream from an imaging area, followed by applying a first imaging pulse train to the imaging area, and (2) applying a control pulse train to a control area followed by applying a second imaging pulse train to the imaging area;
receiving a plurality of first digital data and a plurality of second digital data corresponding respectively to nuclear magnetic resonance (NMR) signals responsive to the first imaging pulse train and to NMR signals responsive to the second imaging pulse train;
forming, from each of the plurality of first digital data, a respective tag image and, from each of the plurality of second digital data, a respective control image;
forming a plurality of perfusion images of the object, each of the perfusion images formed by one of the tag images and a corresponding one of the control images, each perfusion image corresponding to a respective phase offset;
generating a corrected perfusion image by, for respective points of the corrected perfusion image, fitting corresponding points from each of the plurality of perfusion images to a polynomial function; and
outputting the corrected perfusion image to a display, or data storage in a non-transient digital data storage medium, or an outbound data transmission port.
